(19) United States
(12) Patent Application Publication
(10) Pub. No.: US 2018/0250525 A1
(43) Pub. Date: Sep. 6, 2018

(54) METHODS AND DEVICES FOR DISPLACING TISSUES DURING BRACHYTHERAPY

(71) Applicant: Medical Tool and Technology, LLC, Hawthorne, FL (US)

(72) Inventors: John Scott Wheeler, Gainesville, FL (US); Richard David Helmig, Hawthorne, FL (US)

(21) Appl. No.: 15/909,279

(22) Filed: Mar. 1, 2018

Related U.S. Application Data
(63) Continuation-in-part of application No. 62/466,858, filed on Mar. 3, 2017.

(51) Int. Cl. A61N 5/10

(52) U.S. Cl. CPC A61N 5/1014; A61B 2018/0022; A61N 5/1007

(57) ABSTRACT

Tubular constructs attached to a brachytherapy device allowing for the selected displacement of surrounding tissue at a site of radiation application. Tubular constructs have an inflatable balloon portion and an attached non-inflating stem tube. Balloon portions are affixed to the brachytherapy device and inflated with the stem tube to displace tissue away from an area.

### CROSS-REFERENCE TO A RELATED APPLICATION

The present application is a continuation-in-part of U.S. Application Ser. No. 62/466,858, filed Mar. 3, 2017, which is hereby incorporated by reference herein in its entirety, including any figures, tables, or drawings.

### BACKGROUND OF INVENTION

Advancing medicine has allowed for more focused and efficient treatments for cancer. The increasing effectiveness of these treatments has allowed clinicians to turn their attentions to the patient. Now patient safety and comfort can be considered during these delicate cancer treatment procedures.

Brachytherapy is an essential component of the treatment of cervical cancer. Brachytherapy is internal radiotherapy involving placement of a radiation source near, or inside, the tissue to be treated. In treating cervical cancer a hollow applicator is placed adjacent the cervix and a radiation source is fed through the hollow applicator to hold the radiation source close to the affected tissue. There are a number of such hollow applicators that most often take the form of tandem plates or a ring.

Placement of the hollow applicator within the body cavity is uncomfortable for the patient. The clinician must take further measures to prevent movement of the hollow applicator while in the body, to inhibit the radiation from affecting surrounding healthy tissues and organs. The rectum, for example, is malleable and tends to engulf or surround the inserted applicator, thus displacement of the rectum from the vicinity of the applicator is a necessity. Some applicators include a displacement blade that is inserted alongside the applicator to physically displace the rectum away from the radiation source. The blade is extremely uncomfortable for the patient. U.S. Published Patent Application No. 2015/0190621 describes a donut-shaped balloon that is placed around the ring of a brachytherapy applicator. The balloon is inflated to push tissue around the ring away from the radiation source inside the ring. Unilateral displacement by the balloon of all the organs and tissue surrounding the ring is likewise uncomfortable for the patient and is usually unnecessary. Only those tissues in proximity to the radiation source need to be moved, but is often not possible.

As cancer treatments become more refined allowing for the focused treatment of only affected tissues, patient safety and comfort can again become a consideration. A need remains for techniques and devices for displacing tissues and organs during brachytherapy that effectively protects the healthy tissue while aiding in patient comfort.

### BRIEF SUMMARY

In accordance with the invention, the unnecessary unilateral displacement of tissue around a brachytherapy device during radiation treatment is alleviated by providing one or more balloons that can be selectively placed and attached to the brachytherapy device, prior to treatment, and inflated after the brachytherapy device is emplaced. The one or more balloons provide the option of displacing only tissue(s) in proximity to the radiation source(s). At the end of the treatment, the one or more balloons can be deflated prior to removal. Advantageously, displacing only those tissues in proximity to the radiation source can reduce discomfort for the patient and prevent possible inflammation or tearing of healthy tissue that is unnecessarily displaced or moved aside.

In one embodiment, the balloon is part of a flexible tubular construct with at least a portion that is sufficiently elastic or pliable to be inflatable as a balloon. The inflatable balloon portion of the flexible tubular construct can be attached to a surface of the brachytherapy device. In particular, the inflatable balloon portion of the flexible tubular construct can be attached to the ring piece of a tandem ring brachytherapy device. The attached inflatable balloon portion of the tubular construct can be inflated on the brachytherapy device by another portion of the tubular construct, the stem tube, which can be used to force air into the inflatable portion. In a further embodiment, the inflatable portion is removably attached to the brachytherapy surface. This allows the tubular construct to be removed after a treatment allowing the brachytherapy device to be re-sterilized.

It should be noted that this Brief Summary is provided to generally introduce the reader to one or more select concepts described below in the Detailed Disclosure in a simplified form. This Summary is not intended to identify key and/or required features of the claimed subject matter. Other aspects and further scope of applicability of the present invention will also become apparent from the detailed descriptions given herein. It should be understood, however, that the detailed descriptions, while indicating preferred embodiments of the invention, are given by way of illustration only, since various changes and modifications within the spirit and scope of the invention will become apparent from such descriptions. The invention is defined by the claims below.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1 is a bottom perspective view of a brachytherapy applicator with a single balloon which is inflated placed on its face.

FIG. 2 is a bottom perspective view of the brachytherapy applicator of FIG. 1 with a single balloon which is deflated placed on its face.

FIG. 3 is a side elevational view of the brachytherapy applicator of FIG. 1 with a single balloon which is inflated placed on its face.

FIG. 4 is a side elevational view of the brachytherapy applicator of FIG. 1 with a single balloon which is deflated placed on its face.

FIG. 5 is a bottom perspective view of a brachytherapy applicator with two balloons which are deflated placed on its face.

FIG. 6 is a bottom perspective view of the brachytherapy applicator of FIG. 5 with two balloons which are deflated placed on its face.

FIG. 7 is a bottom perspective view of a brachytherapy applicator with a single balloon which are inflated placed on the side of the ring.

FIG. 8 is a bottom perspective view of a brachytherapy applicator with two balloons which are inflated placed on the ring.

FIG. 9 is a bottom perspective view of a brachytherapy applicator with a single balloon which are inflated placed on its face and shows a syringe used to inflate the balloon.

FIG. 10 illustrates one example of a tubular construct with more than one balloon portion on a stem tube.

### DETAILED DISCLOSURE

The subject invention provides a tubular construct with an individual inflatable balloon portion that can be affixed to a brachytherapy applicator. The balloon portion can be affixed anywhere on the brachytherapy applicator, usually on the ring piece of the device, allowing tissue displacement that is specific to radiation placement and treatment vacinity.

The balloon portion of the subject invention can be any inflatable component that can be configured as part of the tubular construct to be inflated and deflated. The tubular construct comprises at least one inflatable balloon portion and a non-inflating extended stem tube used for inflation and deflation of the inflatable balloon portion. While the stem tube can be made from elastic materials that are similar to, or the same as, the inflatable balloon portion, the stem tube is inhibited or prevented from inflating when the inflatable balloon portion inflates. An attachment apparatus positions and secures the balloon portion to the brachytherapy applicator. Preferably, all elements of the tubular construct, including the balloon, stem tube, and attachment apparatus of the subject invention are proved, reliable, and accepted for use by the Federal Drug Administration (FDA).

As used herein, and unless otherwise specifically stated, the terms “operable communication,” “operable connection,” “operably connected,” “cooperatively engaged” and grammatical variations thereof mean that the particular elements are connected in such a way that they cooperate to achieve their intended function or functions. The “connection” or “engagement” may be direct, or indirect, physical or remote.

Additionally, the terms “approximately” and “about” are defined as +/−5% in order to cover manufacturing variances and device tolerances.

Furthermore, reference is made throughout the application to the “proximal end” or “proximal direction” and “distal end” or “distal direction.” As used herein, the proximal end or proximal direction is that end that is closest to or inserted into the body. For example, the balloon portion is at the proximal end of the tubular construct. Conversely, the distal end or distal direction of the device is that end which is typically furthest from the body or that is not inserted into the body. For example, the connector end of the stem tube for attachment to an air injection source can be located at the distal end of the tubular construct.

It is to be understood that the figures and descriptions of embodiments of the present invention have been simplified to illustrate elements that are relevant for a clear understanding of the invention, while eliminating, for purposes of clarity, other elements that may be well known. Those of ordinary skill in the art will recognize that other elements may be desirable and/or required in order to implement the present invention. However, because such elements are well known in the art, and because they do not facilitate a better understanding of the present invention, a discussion of such elements is not provided herein.

The material utilized for a tubular construct will, ideally, be approved for use in the human body or be Generally Regarded As Safe (GRAS). As will be discussed, the balloon portion can be inflated to displace surrounding tissue from the treatment vicinity of the radiation source of the brachytherapy device. The balloon portion can be a non-compliant type of balloon, semi-compliant type of balloon, or compliant type of balloon.

Non-compliant or semi-compliant balloon types typically expand to a specific or pre-determined size, regardless of the internal pressure. A non-compliant or semi-compliant balloon portion can be, but is not limited to, plastic, nylon, polyethylene terephthalate, similar materials, or other materials that is flexible, but not particularly elastic. They can also have a high burst pressure rating.

Compliant balloon types expand as internal pressure increases and can continue to expand until the limit of the balloon material are reached. They can be flexible and also more elastic, also making them more conformable. Materials suitable for use as a compliant balloon portion include, but are not limited to, polyimide, polyester, polyolefin copolymers, fluoropolymers, nature and synthetic rubber, silicon, thermoplastic elastomers, nylon, mylar, polyethelene, latex, and polyvinyl chloride. In a particular embodiment, the inflatable portion is a latex rubber balloon, such as the type used to open veins and arteries during cardiovascular surgery. It is within the skill of a person trained in the art to determine the type of balloon and balloon material for a tubular construct of the subject invention.

It can be beneficial if at least the balloon portion is observable in vivo during placement and/or inflation for more accurate results. In one embodiment, one or more materials of, or on, the balloon portion are radiopaque allowing it to be observable by any of a variety of known medical devices. In an alternative embodiment, the material of a balloon portion is radiolucent, such that the balloon cannot be observed by standard medical devices. With this embodiment, the material of the balloon portion can be compatible with substances that are radiopaque. Thus, the balloon can be filled with a radiopaque material, which can still allow for observation during placement and inflation.

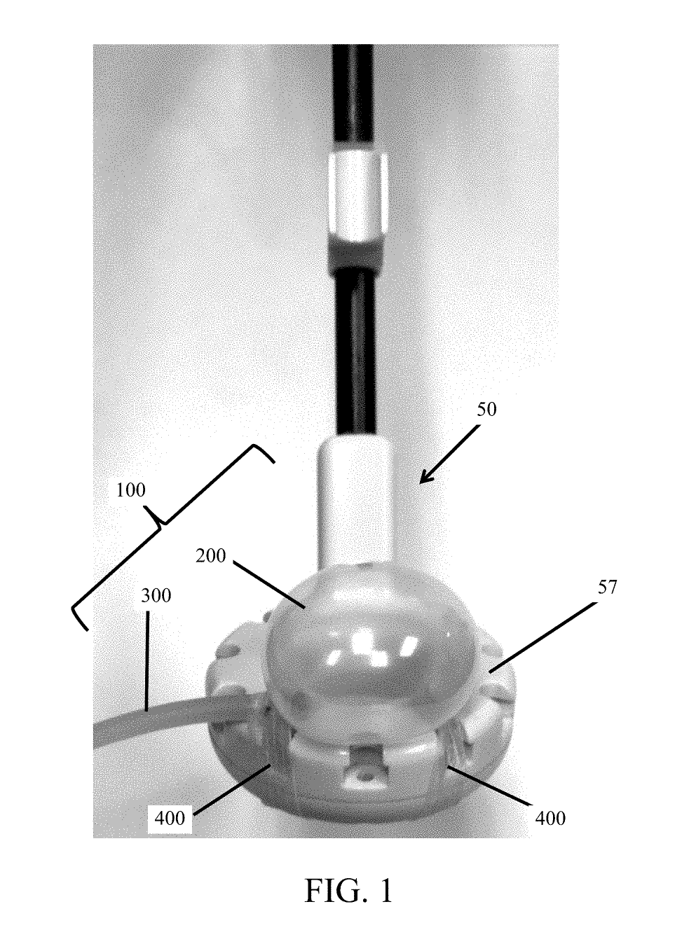

Reference will be made to the attached Figures on which the same reference numerals are used throughout to indicate the same or similar components. With reference to the attached Figures, which show certain embodiments of a the subject invention, it can be seen in FIG. 1 that a tubular construct 100 of the subject invention comprises an inflatable balloon portion 200 and at least one attachment apparatus 400 for selectively and removably affixing at least the balloon portion to the brachytherapy device. Attached to the balloon portion is a stem tube 400 that directs air into the balloon portion for inflation. Additional embodiments can include one or more tabs 250 on the balloon portion operable with the attachment apparatus and connectors 450 that can be used to attach the stem to an air or liquid source 25 for inflating the balloon portion. Each of these general components can have one or more sub-components, which will be discussed in detail below.

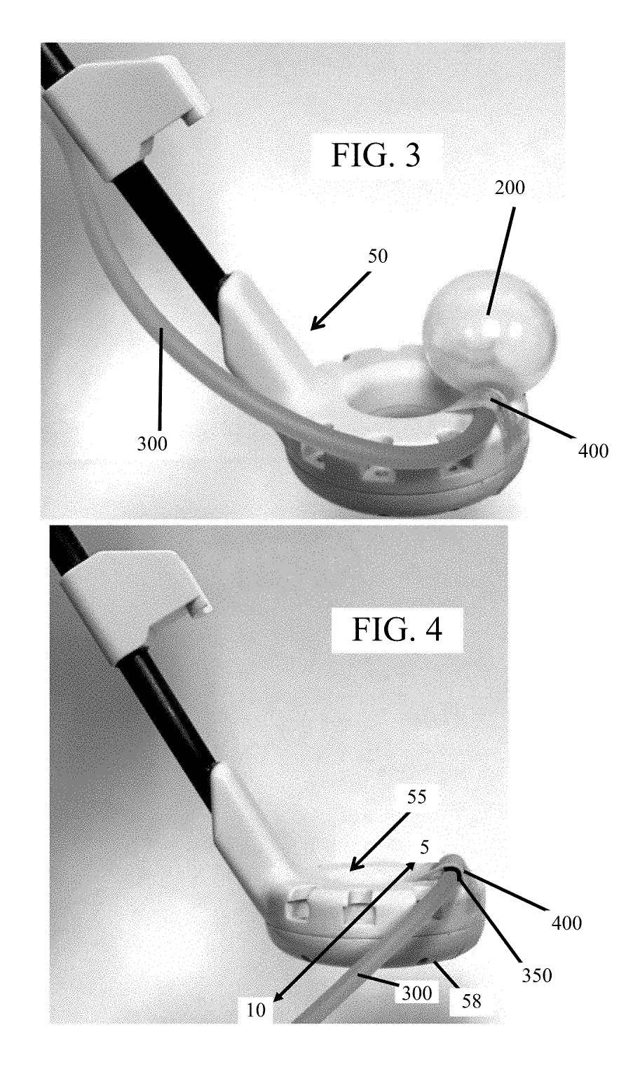

One embodiment of the tubular construct 100 of the subject invention is shown in the FIGS. 1-9, which show the balloon portion attached to a ring piece 55 of a commonly used brachytherapy device 50 to treat cervical cancer. FIG. 1 shows a bottom view of the ring piece. In one embodiment, a balloon portion is placed on the bottom face 57 of the ring piece. In position in the patient, the top face 58 of the ring is placed against the cervix. Radiation is delivered to various positions about the top face of the ring adjacent the cervix. The inflated balloon portion 200 is attached to the ring portion to displace from the treatment vicinity other healthy tissue, such as, for example, the rectum of the patient, distancing the heathy tissue from the radiation source. Once treatment is completed, the balloon portion can be deflated prior to removal from the body.

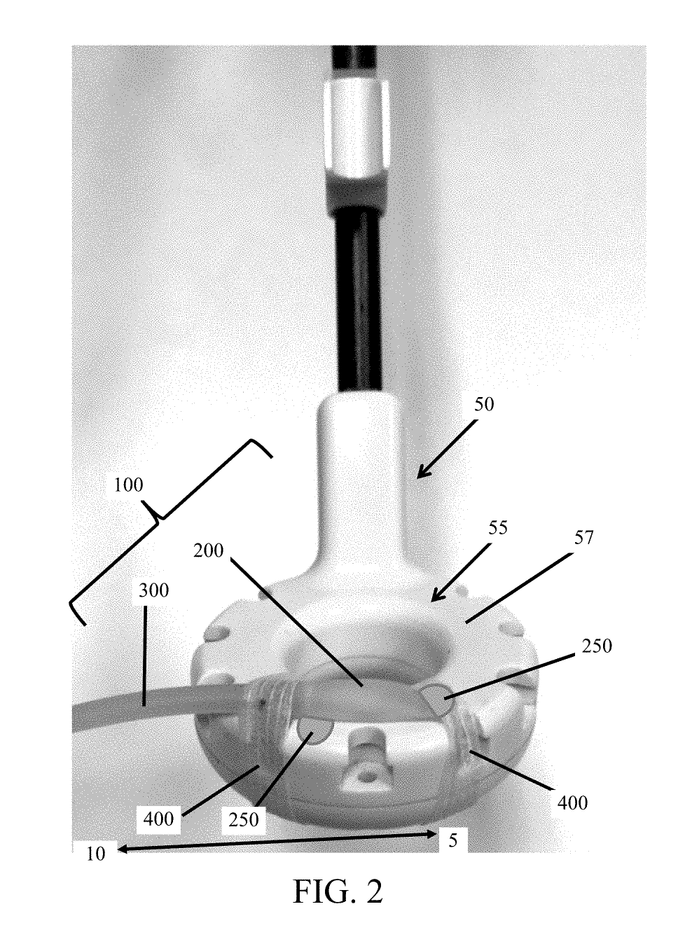

The inflatable balloon portion 200 can be affixed or affixable to the end of a stem tube 300. In one embodiment, the inflatable balloon portion has a thinner wall than that of the stem tube. By way of a non-limiting example, which is shown in FIG. 2, the inflatable balloon portion can be a cardiovascular inflatable material glued to the end of a latex tube with rubber cement. Rubber cement provides a reliable air and liquid tight seal. Other can devices, materials, and techniques can be used for connecting an inflatable portion and stem tube. Such attachment apparatuses 400 can include, but are not limited to, physical connectors, adhesives or adhesive-like materials, adhesive tape, ties (e.g., “zip ties”, medical-grade string or twine, dental floss), ultrasonic or heat welding, crimping, frictional connections, and other methods known to those with skill in the art. In use, the thinner walled balloon portion inflates as air, gas, or liquid passes through the lumen of the thicker walled stem tube. While the tubular construct 100 can be two connected pieces, e.g., an inflatable balloon portion attached to a stem, a tubular construct of the subject invention can be a single- or one-piece device. One skilled in the art would understand how to configure one more materials to form a single- or one-piece tubular construct, with an inflatable balloon portion and a non-inflatable stem tube.

The inflatable balloon portion 200 of a tubular construct 100 can be located at the distal end 5 of the tubular construct. Alternatively, the inflatable balloon portion can be positioned slightly proximal 10 to the distal end. In a further embodiment, there can be a tab 400 disposed at the distal end of the balloon portion. In yet a further embodiment, there can be one or more tabs arranged on other areas of the balloon portion. A tab can provide an anchor point for the inflatable balloon portion and be operable with an attachment apparatus 400. FIGS. 2, 5 and 8 illustrate examples of a balloon portion with one or more tabs thereon operable with an attachment apparatus.

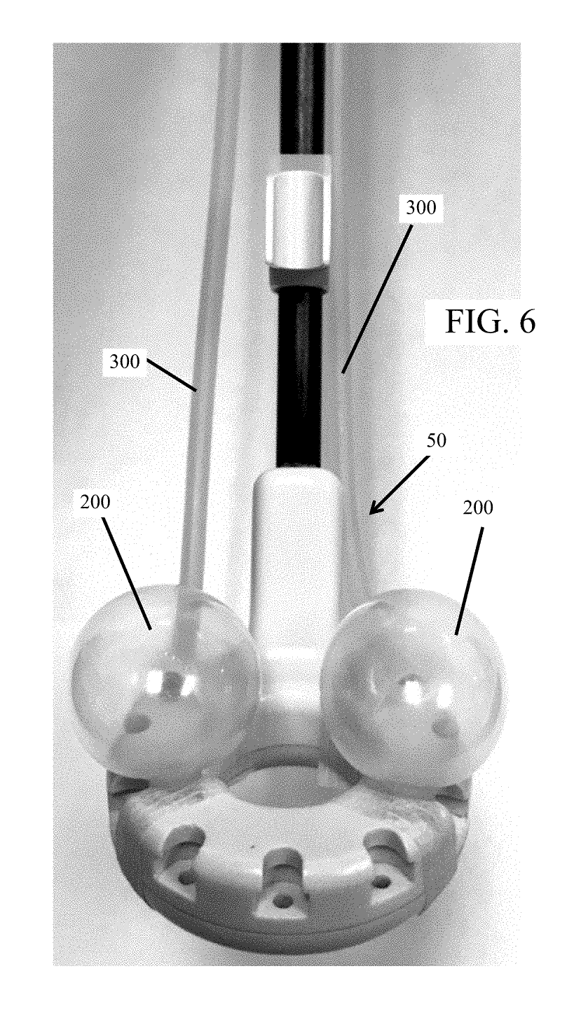

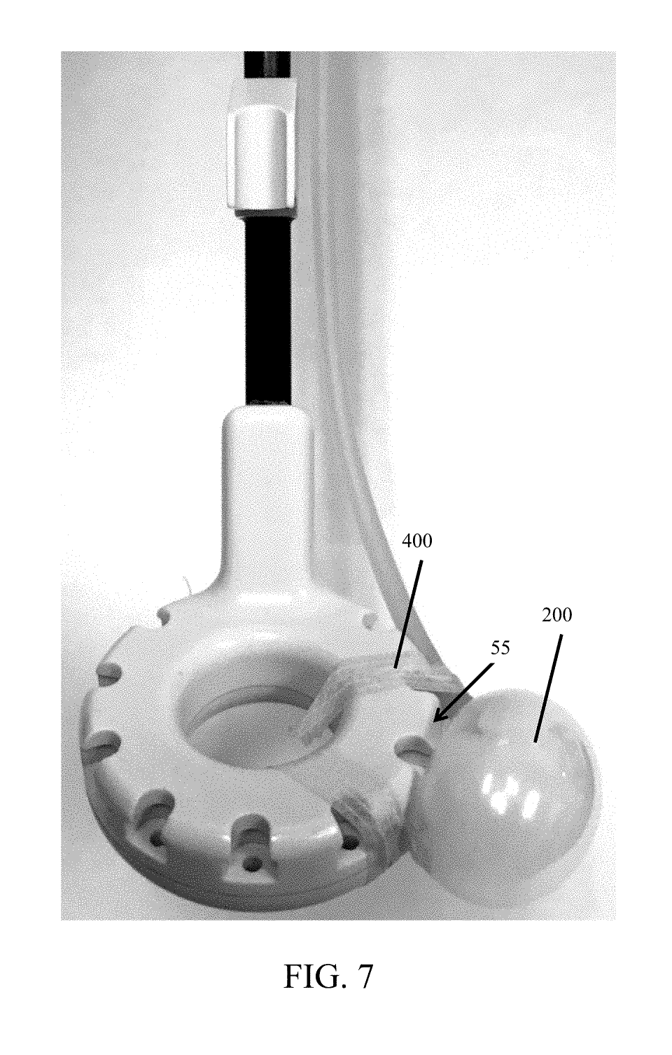

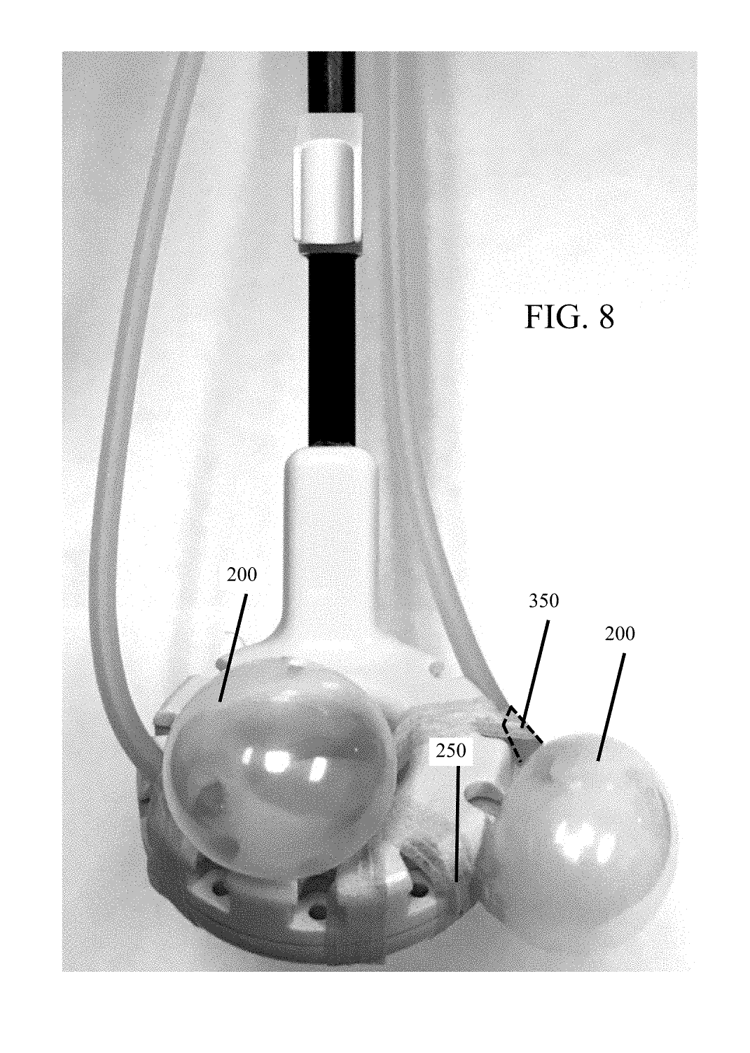

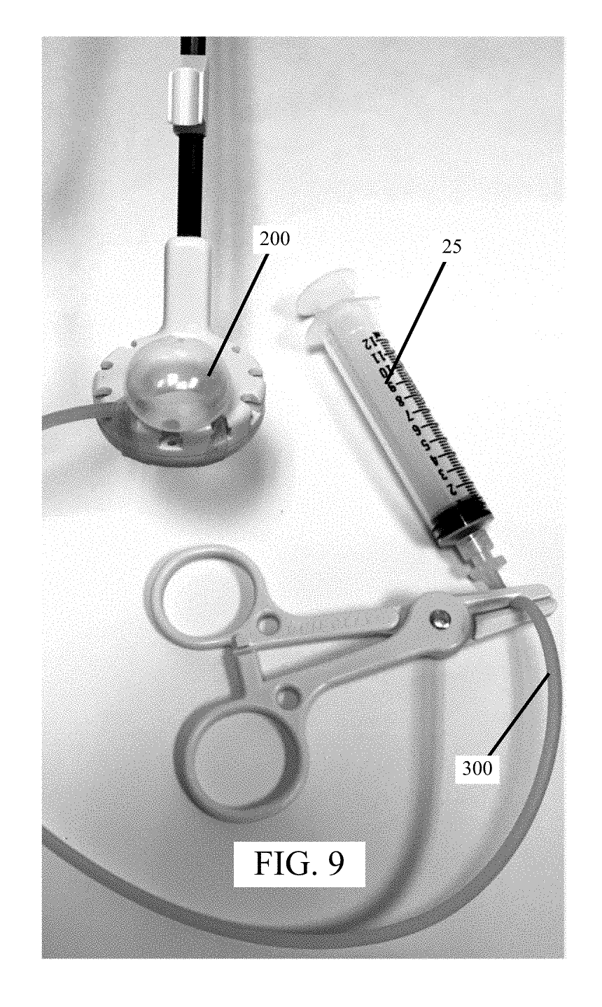

Depending upon the location of the tissue to be treated, it can be necessary to displace more than one area of healthy tissue away from the treatment area. FIGS. 1 and 7 illustrate ring pieces of a brachytherapy device on which a single balloon portion 200 is affixed. More than one inflatable balloon portion can be affixed to different locations on the ring piece, such as shown, for example, in FIGS. 6 and 8.

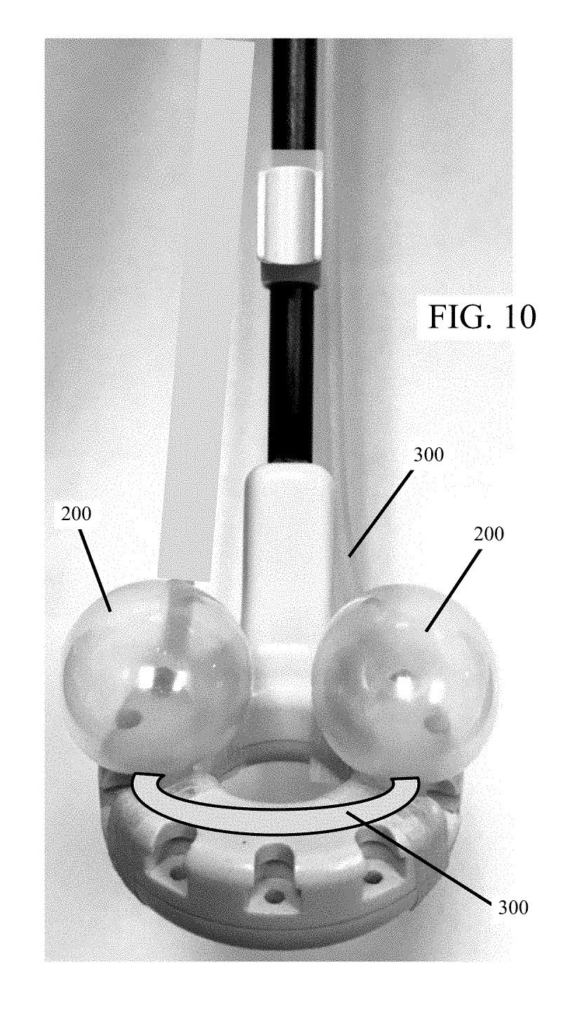

In an alternative embodiment, a tubular construct can have more than one balloon portion disposed along the length of a stem tube 300, allowing a technician to choose inflation positions and tailor balloon positions to patient anatomy. FIG. 10 illustrates one example of a tubular construct with more than one balloon portion 200 at the distal end 5 of a stem tube 300. In this example, there is also shown a portion of the stem tube between the balloon portions, which can vary in length. Inflatable portions that are not needed for a particular patient can be prevented from inflating by covering with a sleeve or placing something on them that inhibits their being inflated, such as, for example, adhesive tape.

Furthermore, the size or length of the inflatable balloon portion can vary. The amount of air forced into the balloon portion 200 can also be used to customize the size of the inflated balloon portion. Thus, if a larger inflated balloon portion is desired, more air can be pumped or forced into the balloon portion. Although the examples shown in the FIGS. have smaller inflatable balloon portions, even smaller or a larger balloon portions may sometimes be useful. Embodiments of the balloon portion have a longitudinal length 15. A longitudinal length is the distance between the distal end 5 and the proximal end 10 of the tubular construct. The longitudinal length of a balloon portion can be about 0.5 cm, 1 cm, 1.5 cm, 2.0 cm, 2.5 cm, 3 cm, 3.5 cm, 4 cm, 4.5 cm, and/or about 5 cm, or any longitudinal length in a range between any two of the listed values.

When the balloon portion 200 is inflated, it can elevate or rise above the surface of the brachytherapy device to which it is attached, such as shown, for example, in FIGS. 1, 7, and 8. The distance 275 that the balloon portion elevates above the surface of the brachytherapy device can depend upon the amount of air or liquid injected into the balloon portion through the stem tube. Embodiments of a balloon portion can elevate, when inflated, above the surface of a brachytherapy device to a distance of approximately 0.1 cm, 0.02 cm, 0.3 cm, 0.4 cm, 0.5 cm, 0.8 cm, 1.0 cm, 1.5 cm, 2.0 cm, 2.5 cm, 3 cm, 3.5 cm, 4 cm, 4.5 cm, 5 cm, 5.5 cm, 6 cm, 6.5 cm, 7 cm, 7.5 cm, and/or 8 cm. and/or any distance in a range between any two of the listed values. In a particular embodiment, the balloon portion elevates about 2.5 cm above the surface 55 of the brachytherapy device. Balloon portions 200 can be attached to the brachytherapy device by balloon attachment apparatuses 400. In a specific embodiment, balloon portions are affixed to the ring piece of the brachytherapy device with adhesive tape, such as, for example, suture tape. Suture tape that has a fabric texture has a strong adhesive and is reinforced with fibers to inhibit tearing. Advantageously, suture tape has also been approved for use on the body by the FDA. It can be preferable for there to be more than one affixing point of the balloon portion to the brachytherapy device. Preferably, the balloon portion is affixed to the brachytherapy device at two points, such as, by way of example, a point on either side of the balloon portion, such as shown, for example, in FIGS. 2 and 7. This can ensure that the balloon portion when inflated does not change position on the device and provides precision tissue displacement and comfort for the patient.

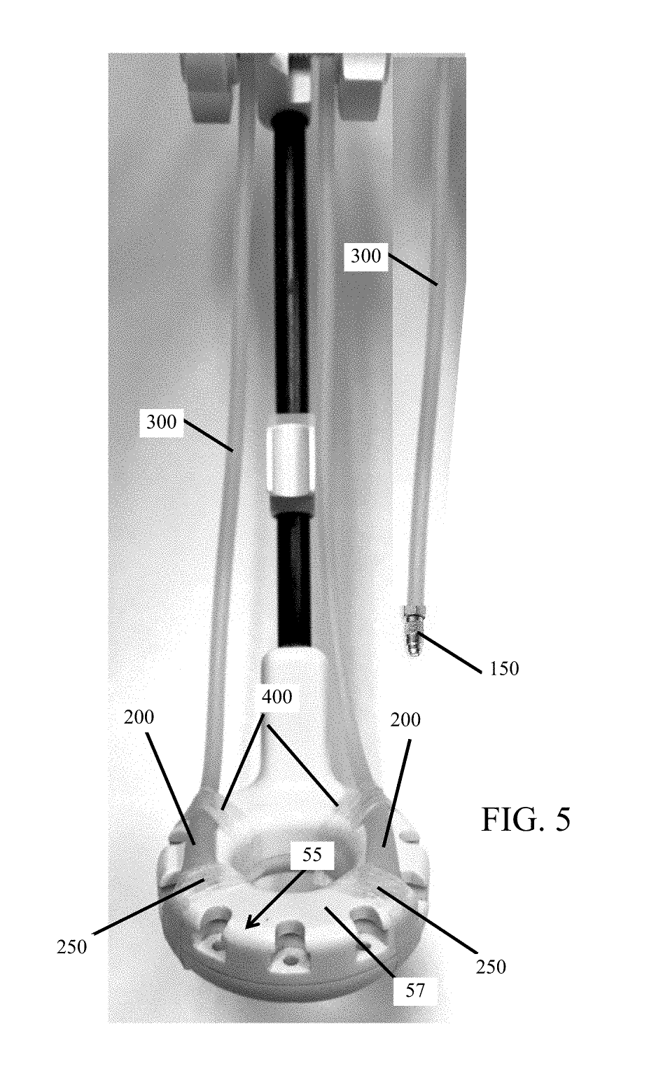

Certain attachment apparatuses 400 can crush, block, reduce or otherwise close the lumen 325 of the stem tube inhibiting reliable inflation of the balloon portion 200, particularly when attachment apparatuses are placed around the stem tube to secure the balloon portion, shown in the examples in FIGS. 5 and 8. With this configuration, the tightness of the suture tape around the ring piece 55 should be carefully monitored and adjusted when placing and applying the balloons on the brachytherapy device to insure proper inflation. To reduce the potential for improper application of an attachment apparatus, butterfly shaped tape can be used where the narrower part of the tape crosses the stem. Alternatively, a stem tube protector 350 can be used on the stem tube. A stem tube protector can be a rigid or semi-rigid component that goes around part or all of the stem tube 300 where it is used to attach the balloon portion 200 to the brachytherapy device. In one embodiment, a stem tube protector is a full or half-ring or sleeve on the outside of the stem tube that can fortify or strengthen an area or portion of the stem tube preventing it from being crushed when an attachment apparatus is used thereon. In an alternative embodiment, a stem tube protector is a full or half-ring or sleeve on the inside of the stem tube than can also fortify or stregthen the stem tube and prevent it from being crushed with an attachment apparatus is placed on the outside of the stem tube. By way of example, one advantage of using the two piece balloon configuration joined by rubber cement is that the rubber cement glue can hardens to provide a type of stem tube protector that keeps the lumen of the stem open when it is taped to the ring piece.

A balloon portion can be inflated with either air, gas, or a liquid. Liquid with an imaging or contrast agent can be used to inflate the balloon portion, assisting the clinician in identifying balloon portion position in relation to the surrounding tissue. In one embodiment, there is a connector 150 operably connected to the proximal end 100 of the stem tube. The connector can be used to attach the stem tube to the source of the air, gas, or liquid that will be used to fill the balloon portion.

FIG. 9 illustrates a tubular construct 100 wherein the stem tube 300 is attached to a syringe 25 to fill a balloon portion. The syringe can have a volume sufficient to fill the balloon and, advantageously, is easy and familiar to manipulate. In one embodiment, the syringe can have a volume that can fill the balloon portion to a desired or pre-determined maximum inflation potential. Furthermore, any of a variety of clamps, valves, or similar fluid control apparatuses can be used on or in-line with the stem tube to trap and/or release the inflating material in the balloon portion. Likewise, other techniques and devices can be used to inflate and maintain air or liquid in a balloon portion. For example, the balloon portion can also be inflated mechanically using pumps and hoses. Certain pumps may allow for a more controlled fill of the balloons that can be inflated to displace tissue as little as 0.1 cm and as much as 8 cm. Clamps, valves, or apparatuses for controlling release of air or liquid can include, for example, luer lock clamps, in-line clamps, ball valves, and other apparatuses known to those of skill in the art.

The balloons of the subject invention allow specific and precise displacement of healthy tissue from a radiation source during brachytherapy. Balloon portions can be placed on several positions on and around the ring applicator of a brachytherapy device. The number and position of the balloons can be varied to accommodate the unique anatomy of each patient. Although the present invention is described for use on a ring brachytherapy device used to treat cervical cancer, it is noted that the simple, reliable means of application of the subject invention allows these balloons to be used on any type of brachytherapy device. The balloons can be placed on other cervical brachytherapy applicators like the tandem and ovoid applicator or a vaginal applicator. They can likewise be applied to other applicator devices of intracavity brachytherapy used to treat lung, prostate, or colon cancer. The selective displacement of non-target tissue provided by the balloons of the subject invention insure the comfort and safety of a patient.

It is understood that the foregoing examples are merely illustrative of the present invention. Certain modifications of the articles and/or methods may be made and still achieve the objectives of the invention. Such modifications are contemplated as within the scope of the claimed invention.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

The examples and embodiments described herein are for illustrative purposes only and that various modifications or changes in light thereof will be suggested to persons skilled in the art and are to be included within the spirit and purview of this application.

Any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A kit for displacing tissue, in the treatment vicinity of a brachytherapy device, comprising
a tubular construct comprising;
a balloon portion,
at least one tab extending from the balloon portion for attaching to the brachytherapy device,
a stem tube operably attached to the balloon portion for inflating the balloon portion, and
an attachment apparatus for attaching the at least one tab to the brachytherapy device.

2. The kit, according to claim 1, wherein the attachment apparatus is adhesive tape.

3. The kit, according to claim 1, further comprising a stem tube protector.

4. The kit, according to claim 3, wherein the stem tube protector is a sleeve.

5. The kit, according to claim 4, wherein the sleeve is on the outside of the stem tube.

6. The kit, according to claim 1, wherein the balloon portion comprises at least one radiopaque material.

7. The kit, according to claim 1, wherein the balloon portion has a longitudinal length of between about 0.5 cm to about 5 cm.

8. The kit, according to claim 7, wherein the balloon portion has a longitudinal length of between about 2 cm and about 3 cm.

9. The kit, according to claim 1, wherein the balloon portion, when attached to the surface of the brachytherapy device, elevates to a distance of between about 0.1 cm and about 8 cm.

10. The kit, according to claim 9, wherein the balloon portion, when attached to the surface of the brachytherapy device, elevates to a distance of between about 0.1 cm to about 2.5 cm.

11. A method for displacing tissue, in the treatment vicinity of a brachytherapy device, comprising:
positioning relative to the brachytherapy device a tubular construct comprising,
a balloon portion, for contacting the surface of the brachytherapy device;
at least one tab extending from the balloon portion for attaching to the surface of the brachytherapy device, to secure the balloon portion on the brachytherapy device; and
a stem tube operably attached to the balloon portion for inflating the balloon portion,
securing to the surface of the brachytherapy device the at least one tab; and
utilizing the stem tube to inflate the balloon portion.

12. The method, according to claim 11 further comprising placing the brachytherapy device with the balloon portion attached thereto in the vicinity of a treatment area prior to inflating the balloon portion.

13. The method, according to claim 12, further comprising utilizing a medical device to observe inflation of the balloon portion.

14. The method, according to claim 13, wherein the balloon portion comprises at least one radiopaque material.

15. The method, according to claim 13, wherein the balloon portion is inflated with a radiopaque material.

16. The method, according to claim 11, further comprising a stem protector on the stem tube and the method further comprises securing the stem protector to the surface of the brachytherapy device.

17. A tubular construct, adapted to be attached to a brachytherapy device, comprising:
a balloon portion;
at least one tab extending from the balloon portion for attaching to the brachytherapy device; and
a stem tube operably attached to the balloon portion for inflating the balloon portion.

18. The tubular construct, according to claim 17, further comprising a stem tube protector.

19. The tubular construct, according to claim 17, further comprising an attachment apparatus for attaching at least the at least one tab to the brachytherapy device.

20. The tubular construct, according to claim 19, wherein the attachment apparatus comprises an adhesive tape.
